(19) United States
(12) Patent Application Publication
(10) Pub. No.: US 2026/0335593 A1
(43) Pub. Date: Oct. 29, 2026

(54) Split Tip Catheter Placement Systems and Methods

(71) Applicant: Pristine Access Technologies LTD., Franklin Lakes, NJ (US)

(72) Inventors: Brenda L. F. Fedor, Holladay, UT (US); Andrzej J. Chanduszko, Chandler, AZ (US)

(21) Appl. No.: 19/490,327

(22) PCT Filed: Jun. 5, 2023
(86) PCT No.: PCT/US2023/024494

§ 371 Date: Dec. 4, 2025

(51) Int. Cl. A61M 25/00; A61M 25/01

(52) U.S. Cl. CPC A61M 25/0068; A61M 2025/0177

(57) ABSTRACT

Catheter placement systems for split tip catheters can include a first guidewire, a second guidewire and a link slidably engaged with each. As the first guidewire is a withdrawn proximally, a stopping feature urges the link towards the split tip feature, transitioning the split tip feature from a Y-shape to a placement configuration, extending parallel to the axis of the catheter. Embodiments include a rod engaged with a first guidewire and a sheath extending proximally to retain the split tip feature in a placement configuration. The rod and sheath can then be withdrawn proximally through the first lumen to disengage the catheter. Embodiments include a magnetic element configured to secure a distal tip of a first guidewire with a distal tip of a second guidewire to retain the split tip feature in a placement configuration.

### BACKGROUND

Briefly summarized, embodiments of the present invention are directed to catheter placement systems and associated methods thereof. Split tip catheters facilitate withdrawing a blood flow from a first position while providing a return flow at a different position to ensure little or no mixing of fluids therebetween. The distal tips of these catheters can include various flow modifying structures to facilitate the flow path and mitigate the formation of thrombosis or the like. However, placing such delicate and complex tip structures can be challenging. The multiple distal tips can be highly flexible and can be in a spaced apart configuration to mitigate mixing of the different fluid flows.

What is needed therefore is a system and method to support the distal tips in a configuration that allows for negotiating tortuous vascular pathways to a target location. Once positioned at the target location the supporting structures need to disengage the catheter and be removed with little or no disturbance to the position of the catheter. Disclosed herein are systems and methods that address the foregoing.

### SUMMARY

In some aspects, the techniques described herein relate to a catheter placement system including, a split tip catheter having a catheter body extending along a longitudinal axis and defining a first lumen and a second lumen, and a split tip feature including, a first split tip end extending at a first angle relative to the longitudinal axis and including a distal portion of the first lumen encircled by a first outer wall, and a second split tip end extending at a second angle relative to the longitudinal axis and including a distal portion of the second lumen encircled by a second outer wall, a first guidewire disposed in the first lumen and including a stop feature disposed on a distal portion thereof, a second guidewire disposed in the second lumen, and a link defining a first aperture configured to receive a portion of the first guidewire, and a second aperture configured to receive a portion of the second guidewire.

In some aspects, the techniques described herein relate to a catheter placement system, wherein the stop feature includes an abutment surface configured to engage the link and retain the link to the first guidewire.

In some aspects, the techniques described herein relate to a catheter placement system, wherein the stop feature includes a barb having a tapered surface facing distally and the abutment surface facing proximally and configured to slidably engage the first aperture of the link in a first direction and to prevent the first guidewire disengaging the first aperture of the link in a second direction.

In some aspects, the techniques described herein relate to a catheter placement system, wherein the link, engaged with both the first guidewire and the second guidewire, elastically deforms the split tip feature to a placement configuration where the first split tip end and the second split tip end extend substantially parallel to the longitudinal axis.

In some aspects, the techniques described herein relate to a catheter placement system, wherein the first angle extends in a first direction relative to the longitudinal axis, and the second angle extends in a second direction relative to the longitudinal axis.

In some aspects, the techniques described herein relate to a method of placing a split tip catheter having a catheter body extending along a longitudinal axis and defining a first lumen and a second lumen, and a split tip feature including a first split tip end extending at a first angle and a second split tip end extending at a second angle, relative to the longitudinal axis, the method including, slidably engaging a distal portion of a first guidewire with a first aperture of a link, slidably engaging a distal portion of a second guidewire with a second aperture of the link, the first guidewire disposed within the first lumen and the second guidewire disposed within the second lumen of the split tip catheter, withdrawing the first guidewire proximally through the first lumen, engaging a stop feature disposed at a distal end of the first guidewire with the link to urge the link proximally towards the first split tip end, elastically deforming the first split tip end and the second split tip end to extend substantially parallel with the longitudinal axis, and advancing the split tip catheter over the second guidewire to a target location.

In some aspects, the techniques described herein relate to a method, further including, withdrawing the second guidewire proximally to disengage the second aperture, transitioning the first split tip end to extend at the first angle and the second split tip end to extend at the second angle, withdrawing the first guidewire proximally through the first lumen, and urging the link proximally through the first lumen.

In some aspects, the techniques described herein relate to a method, wherein the stop feature includes a tapered surface facing distally and is configured to slidably engage the first aperture of the link in a first direction, and an abutment surface facing proximally and configured to prevent the link from disengaging the first guidewire in a second direction, opposite the first direction.

In some aspects, the techniques described herein relate to a method, wherein the first angle extends between 45° and 60° in a first direction from the longitudinal axis, and the second angle extends between 45° and 60° in a second direction from the longitudinal axis, the second direction being opposite the first direction.

In some aspects, the techniques described herein relate to a catheter placement system including, a split tip catheter having a catheter body extending along a longitudinal axis and defining a first lumen and a second lumen, and a split tip feature including, a first split tip end extending at a first angle relative to the longitudinal axis in a first direction and including a distal portion of the first lumen encircled by a first outer wall, and a second split tip end extending at a second angle relative to the longitudinal axis in a second direction, opposite the first direction, and including a distal portion of the second lumen encircled by a second outer wall, a first guidewire disposed in the first lumen, and including a first magnetic element disposed at a distal end thereof, and a second guidewire disposed in the second lumen, wherein the first magnetic element secures a distal portion of the first guidewire to a distal portion of the second guidewire to elastically deform the first split tip end adjacent the second split tip end.

In some aspects, the techniques described herein relate to a catheter placement system, wherein the second guidewire includes a second magnetic element disposed at a distal end thereof.

In some aspects, the techniques described herein relate to a catheter placement system, wherein one or both of the first magnetic element or the second magnetic element includes a permanent magnet.

In some aspects, the techniques described herein relate to a method of placing a split tip catheter having a split tip feature including a first split tip end extending a first angle to a longitudinal axis of the split tip catheter, and a second split tip end extending at a second angle to the longitudinal axis, the method including, placing a first guidewire in a first lumen of the split tip catheter, the first guidewire including a first magnetic element disposed at a distal end thereof, a distal tip of the first guidewire disposed distally of a distal tip of the first split tip end, placing a second guidewire in a second lumen of the split tip catheter, a distal tip of the second guidewire disposed distally of a distal tip of the second split tip end, engaging the first magnetic element with a distal portion of the second guidewire, elastically deforming the first split tip end and the second split tip end to extend parallel to the longitudinal axis, and advancing the first guidewire, the second guidewire and the split tip catheter to a target location.

In some aspects, the techniques described herein relate to a method, further including, withdrawing the first guidewire proximally from the first lumen to disengage the first magnetic element from the distal portion of the second guidewire, transitioning the first split tip end to extend at the first angle and the second split tip end to extend at the second angle, and withdrawing the second guidewire proximally from the second lumen.

In some aspects, the techniques described herein relate to a method, wherein the first angle extends in a first direction from the longitudinal axis, and the second angle extends in a second direction from the longitudinal axis, opposite the first direction.

In some aspects, the techniques described herein relate to a catheter placement system including, a split tip catheter having a catheter body extending along a longitudinal axis and defining a first lumen and a second lumen, and a split tip feature including, a first split tip end extending at a first angle relative to the longitudinal axis in a first direction and including a distal portion of the first lumen encircled by a first outer wall, and a second split tip end extending at a second angle relative to the longitudinal axis in a second direction, opposite the first direction, and including a distal portion of the second lumen encircled by a second outer wall, a first magnetic element disposed in the distal portion of the first lumen, and a second magnetic element disposed in the distal portion of the second lumen and configured to magnetically attract the first magnetic element to elastically deform the first split tip end adjacent the second split tip end.

In some aspects, the techniques described herein relate to a catheter placement system, wherein the first magnetic element includes a first tether extending proximally through the first lumen, and the second magnetic element includes a second tether extending proximally through the second lumen.

In some aspects, the techniques described herein relate to a method of placing a split tip catheter having a catheter body defining a first lumen and a second lumen, and a split tip feature including a first split tip end extending a first angle to a longitudinal axis of the split tip catheter, and a second split tip end extending at a second angle to the longitudinal axis, the method including, placing a first magnetic element with a lumen of the first split tip end, placing a second magnetic element with a lumen of the second split tip end, engaging the first magnetic element with the second magnetic element, elastically deforming the first split tip end and the second split tip end to extend parallel to the longitudinal axis, and advancing the first magnetic element, the second magnetic element and the split tip catheter to a target location.

In some aspects, the techniques described herein relate to a method, further including, urging a first tether coupled to the first magnetic element proximally to withdraw the first magnetic element proximally from the first lumen, transitioning the first split tip end to extend at the first angle and the second split tip end to extend at the second angle, and urging a second tether coupled to the second magnetic element proximally to withdraw the second magnetic element proximally from the second lumen.

In some aspects, the techniques described herein relate to a method, wherein the first angle extends in a first direction from the longitudinal axis, and the second angle extends in a second direction from the longitudinal axis, opposite the first direction.

In some aspects, the techniques described herein relate to a sheath catheter placement system including, a split tip catheter having a catheter body extending along a longitudinal axis and defining a first lumen and a second lumen, and a split tip feature including, a first split tip end extending at a first angle relative to the longitudinal axis in a first direction and including a distal portion of the first lumen encircled by a first outer wall, and a second split tip end extending at a second angle relative to the longitudinal axis in a second direction, opposite the first direction, and including a distal portion of the second lumen encircled by a second outer wall, a rod having a shaft extending longitudinally and a loop disposed at a distal end thereof, the rod disposed in the first lumen and the loop disposed distally of the first split tip end, a sheath configured to invert between a first position and a second position, the sheath in the first position extending proximally from the loop and enclosing both the first split tip end and the second split tip end, the sheath in the second position extending distally of the loop, and a guidewire disposed in the second lumen and slidably engaged with the loop.

In some aspects, the techniques described herein relate to a sheath catheter placement system, wherein the sheath in the first position elastically deforms the first split tip end and the second split tip end to extend parallel to the longitudinal axis.

In some aspects, the techniques described herein relate to a sheath catheter placement system, wherein an assembly of the rod, the split tip catheter and the sheath in the first position is slidably engageable with the guidewire.

In some aspects, the techniques described herein relate to a sheath catheter placement system, wherein the sheath in the second position disengages the first split tip end and the second split tip end and the first split tip end transitions to the first angle and the second split tip end transitions to the second angle.

In some aspects, the techniques described herein relate to a sheath catheter placement system, wherein the sheath in the second position is collapsible and configured to be withdrawn proximally through the first lumen.

In some aspects, the techniques described herein relate to a method of placing a split tip catheter having a first split tip end and a second split tip end, the method including, placing a rod in a first lumen of the split tip catheter, the rod including a shaft and a loop coupled to a distal end thereof, the loop disposed distally of the first split tip end, placing a guidewire in a second lumen of the split tip catheter, extending a distal portion of the guidewire through the loop, extending a sheath over the first split tip end and the second split tip end, a first end of the sheath engaged with an outer edge of the loop and a second end of the sheath disposed proximally of the first end, the sheath elastically deforming the first split tip end from a first angle to extend parallel to a longitudinal axis, and elastically deforming the second split tip end from a second angle to extend parallel to the longitudinal axis, and advancing the split tip catheter, the rod, and the sheath over the guidewire to a target location.

In some aspects, the techniques described herein relate to a method, further including, withdrawing the guidewire proximally from the loop and from the second split tip end, withdrawing the rod proximally to withdraw the loop into the first lumen, inverting the sheath to a second position wherein the second end of the sheath is disposed distally of the first end, and withdrawing the sheath proximally through the first lumen.

In some aspects, the techniques described herein relate to a method, further including transitioning the first split tip end from extending parallel to extend at the first angle and transitioning the second split tip end from extending parallel to extend at the second angle.

In some aspects, the techniques described herein relate to a method, wherein the first angle extends in a first direction from the longitudinal axis and the second angle extends in a second direction from the longitudinal axis, opposite the first direction.

In some aspects, the techniques described herein relate to a catheter placement system including, a split tip catheter having a first lumen and a second lumen, and a split tip feature including, a first split tip end extending at a first angle relative to a longitudinal axis and including a distal portion of the first lumen encircled by a first outer wall, the first split tip end including a first self-closing slit disposed in a side wall thereof, and a second split tip end extending at a second angle relative to the longitudinal axis and including a distal portion of the second lumen encircled by a second outer wall, the second split tip end including a second self-closing slit disposed in a side wall thereof, and a guidewire disposed in a proximal portion of the first lumen and extending through the first self-closing slit and the second self-closing slit, and extending through a distal end of the second lumen.

In some aspects, the techniques described herein relate to a catheter placement system, wherein the first self-closing slit and the second self-closing slit each form a fluid tight seal when the guidewire is removed therefrom.

In some aspects, the techniques described herein relate to a catheter placement system, wherein the guidewire engaged with the first self-closing slit and the second self-closing slit elastically deforms the first split tip end from the first angle to extend parallel with a longitudinal axis of the split tip catheter, and elastically deforms the second split tip end from the second angle to extend parallel with the longitudinal axis of the split tip catheter.

In some aspects, the techniques described herein relate to a catheter placement system, wherein the first angle extends between 45° and 60° in a first direction from the longitudinal axis, and the second angle extends between 45° and 60° in a second direction, opposite the first direction, from the longitudinal axis.

In some aspects, the techniques described herein relate to a catheter placement system, wherein one or both of the first self-closing slit or the second self-closing slit includes a first slit line, and a second slit line intersecting the first slit line at an angle thereto.

In some aspects, the techniques described herein relate to a method of placing a split tip catheter having a first split tip end and a second split tip end including, extending a proximal portion of a guidewire through a second lumen of the split tip catheter, extending a distal portion of the guidewire through a second self-closing slit disposed in a side wall of the second split tip end, extending the distal portion of the guidewire through a first self-closing slit disposed in a side wall of the first split tip end, elastically deforming the first split tip end from a first angle to extend parallel to a longitudinal axis, and elastically deforming the second split tip end from a second angle to extend parallel to the longitudinal axis of the split tip catheter, and advancing the split tip catheter over the guidewire to a target location.

In some aspects, the techniques described herein relate to a method, further including, withdrawing the guidewire proximally to disengage the first self-closing slit and the second self-closing slit, forming a fluid tight seal at the first self-closing slit and the second self-closing slit, and transitioning the first split tip end from extending parallel to extending at the first angle, the second split tip end from extending parallel to extending at the second angle.

In some aspects, the techniques described herein relate to a method, wherein the first angle extends in a first direction from the longitudinal axis, and the second angle extends in a second direction from the longitudinal axis, opposite the first direction.

In some aspects, the techniques described herein relate to a snare catheter placement system including, a split tip catheter having a first lumen and a second lumen, and a split tip feature including, a first split tip end including a distal portion of the first lumen encircled by a first outer wall, and a second split tip end including a distal portion of the second lumen encircled by a second outer wall, a snare having a shaft and a loop disposed at a distal end thereof, the snare disposed in the first lumen and the loop disposed distally of the first split tip end, and a guidewire disposed in the second lumen and extending through the loop.

In some aspects, the techniques described herein relate to a snare catheter placement system, wherein the guidewire engaged with the loop elastically deforms the first split tip end from a first angle to extend parallel to a longitudinal axis, and elastically deforms the second split tip end from a second angle to extend parallel to the longitudinal axis.

In some aspects, the techniques described herein relate to a snare catheter placement system, wherein the first angle extends in a first direction from the longitudinal axis, and the second angle extends in a second direction, opposite the first direction, from the longitudinal axis.

In some aspects, the techniques described herein relate to a snare catheter placement system, wherein an assembly of the snare and the split tip catheter is slidably engageable with the guidewire.

In some aspects, the techniques described herein relate to a method of placing a split tip catheter having a first split tip end and a second split tip end including, placing a snare having a shaft and a loop in a first lumen of the split tip catheter, the loop disposed distally of the first split tip end, placing a guidewire in a second lumen of the split tip catheter, extending a distal portion of the guidewire through the loop, elastically deforming the first split tip end from a first angle to extend parallel to a longitudinal axis, elastically deforming the second split tip end from a second angle to extend parallel to the longitudinal axis, and advancing the split tip catheter and the snare over the guidewire to a target location.

In some aspects, the techniques described herein relate to a method, further including, withdrawing the guidewire from the loop and from the second split tip end, withdrawing the snare proximally to withdraw the loop into the first lumen, and transitioning the first split tip end from extending parallel to extending at the first angle, the second split tip end from extending parallel to extending at the second angle.

In some aspects, the techniques described herein relate to a method, wherein the first angle extends in a first direction from the longitudinal axis, and the second angle extends in a second direction from the longitudinal axis, opposite the first direction.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of a split tip catheter, in accordance with embodiments herein.

FIG. 1B shows a close-up detail of the split tip catheter of FIG. 1A, in accordance with embodiments herein.

FIG. 2A shows a side view of a link catheter placement system, in accordance with embodiments herein.

FIG. 2B shows a distal end view of the system of FIG. 2A with the link shown in wire frame, in accordance with embodiments herein.

FIG. 3A shows a side view of a magnetic catheter placement system, in accordance with embodiments herein.

FIG. 3B shows a side view of a magnetic catheter placement system, in accordance with embodiments herein.

FIG. 4A shows a side view of a sheath catheter placement system in a first position, in accordance with embodiments herein.

FIG. 4B shows a side view of a sheath catheter placement system in a second position, in accordance with embodiments herein.

FIG. 5A shows a side view of a threaded catheter placement system, in accordance with embodiments herein.

FIGS. 5B-5C show close-up detail of a catheter of the threaded catheter placement system of FIG. 5A, in accordance with embodiments herein.

FIG. 6 shows a side view of a snare catheter placement system, in accordance with embodiments herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention, and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

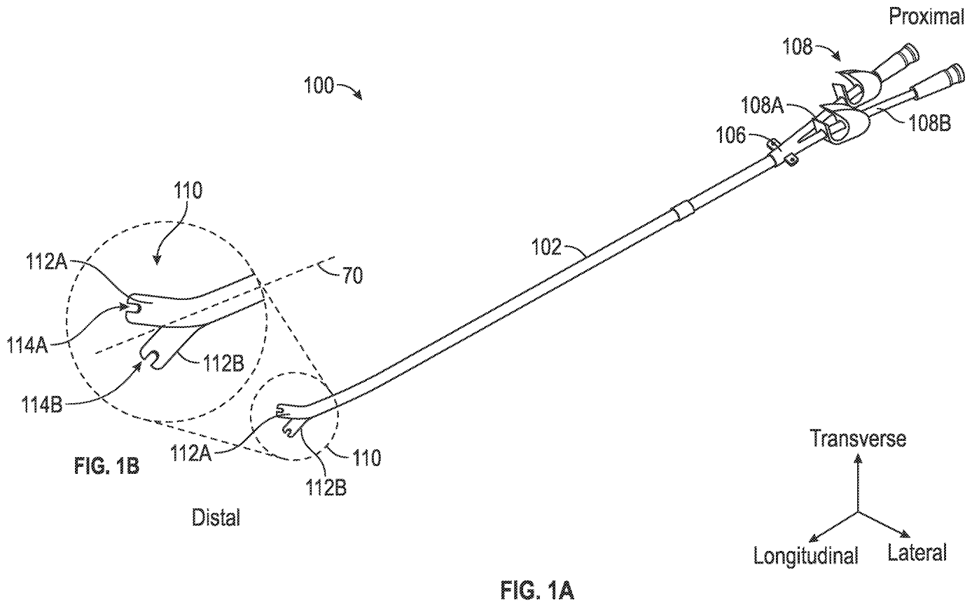

To assist in the description of embodiments described herein, as shown in FIG. 1A, a longitudinal axis extends substantially parallel to an axial length of the catheter. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

Referring to FIGS. 1A-1B, an illustrative embodiment of a split tip catheter (“catheter”) 100 is shown. As will be appreciated, the split tip catheter 100 is exemplary and not intended to be limiting and embodiments disclosed herein can be used with various single lumen catheters, multi-lumen catheters, dialysis catheters, split tip catheters, central venous catheters (CVC), peripherally inserted central catheters (PICC), midline catheters, or the like. In an embodiment the catheter 100, or portions thereof, can be formed from a plastic, polymer, elastomer, or similar suitable biocompatible material. In an embodiment, the catheter 100, or portions thereof, can display flexible mechanical characteristics.

The catheter 100 includes a catheter body 102 defining one or more lumen 104, for example a first lumen 104A and a second lumen 104B. The catheter body 102 is supported at a proximal end by a hub 106 and can include one or more extension legs 108 extending proximally from the hub 106. Each extension leg 108 is in fluid communication with a lumen of the catheter body 102. For example, a first extension leg 108A is in fluid communication with a first lumen 104A, and a second extension leg 108B is in fluid communication with a second lumen 104B.

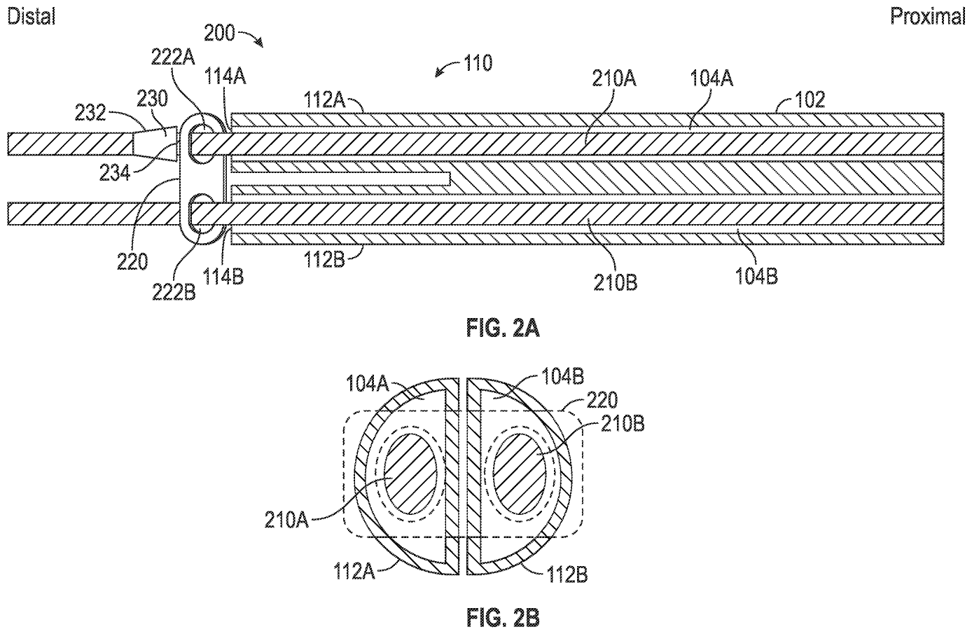

A distal end of the catheter 100 includes a split tip feature 110 including a first split tip end 112A and a second split tip end 112B. The first split tip end 112A includes a distal portion of the first lumen 104A and is defined by an outer wall extending annularly, and the second split tip end 112B includes a distal portion of the second lumen 104B and is defined by an outer wall extending annularly. In an embodiment, as shown in FIG. 2B, one or both of the first split tip end 112A and the second split tip end 112B defines a semi-circular, or “D” shaped cross-sectional outer profile. However, it will be appreciated that one or both of the first split tip end 112A and the second split tip end 112B can define other regular or irregular closed-curve cross-sectional shapes.

In an embodiment, one or both of the first split tip end 112A and the second split tip end 112B includes an opening at a distal tip communicating with a lumen of the catheter. For example, a first distal opening 114A communicates with a first lumen 104A and a second distal opening 114B communicates with a second lumen 104B. In an embodiment, one or both of the first distal opening 114A and the second distal opening 114B can include one or more flow modifying structures configured to modify a fluid flow passing through the opening. Exemplary flow features can include notches, fins, protrusions, or the like.

In an embodiment, as shown in FIG. 1B, one or both of the first split tip end 112A and the second split tip end 112B extends at an angle relative to a central longitudinal axis 70 defined by the catheter body 102. In an embodiment, one or both of the first split tip end 112A and the second split tip end 112B extends at an angle of between 45° and 60° relative to a central longitudinal axis 70. In an embodiment, one or both of the first split tip end 112A and the second split tip end 112B extends at an angle of substantially 45° relative to a central longitudinal axis 70, however greater or lesser angles are also contemplated. In an embodiment, the first split tip end 112A extends in a first direction, and the second split tip end 112B can extend in a second direction. In an embodiment, the first direction and the second direction can be opposite each other, across the central longitudinal axis 70 such that the split tip feature 110 defines a “Y”-shape.

In an embodiment, one or both of the first split tip end 112A and the second split tip end 112B are elastically deformable from the Y-shape. In an embodiment, the Y-shaped split tip feature 110 can be elastically deformable to a placement configuration where the first split tip end 112A and the second split tip end 112B are aligned substantially parallel with the longitudinal axis 70 of the catheter body 102. In an embodiment, in the placement configuration a semi-circular cross-section shape of the first split tip end 112A is aligned with a semi-circular cross-section shape of the second split tip end 112B to define a substantially circular or cylindrical outer profile to the split tip feature 110 (e.g. FIG. 2B). Advantageously, this allows the split tip feature 110 to negotiate tortuous vascular pathways to a target location within the vasculature. Once the split tip feature is positioned at the target location, the split tip feature 110 can resume the elastically undeformed Y-shape.

FIGS. 2A-2B show a link catheter placement system (“system”) 200 for placing the catheter 100. FIG. 2A shows a side view of the system 200. FIG. 2B shows a distal end view of the system 200 with the link 220 shown in wire frame for ease of illustration. The link catheter placement system 200 generally includes a catheter 100 having a split tip feature 110 as described herein, a first guidewire 210A disposed in a first lumen 104A, a second guidewire 210B disposed in a second lumen 104B, and a link 220 defining a first aperture 222A and a second aperture 222B. In an embodiment, the link 220 includes a metal, alloy, plastic, polymer, composite, or the like. In an embodiment, the link 220 is formed of a solid piece of material including a plastic, polymer, metal, alloy, composite, or the like. In an embodiment, the link 220 is formed of one or more strands of material that can be woven or twisted and form a “figure eight” shape, these and other shaped links 220 that include a first aperture 222A and a second aperture 222B are contemplated to fall within the scope of the present invention. In an embodiment, the guidewire 210 can be formed of a plastic, polymer, metal, alloy, stainless steel (SS), shape memory alloy, super-elastic alloy, nitinol, composite, carbon-fiber composite, or the like.

In an embodiment, a distal end of the first guidewire 210A and/or a distal end of the second guidewire 210B and a can extend distally of the distal end of the catheter 100. The link 220 is disposed distally of the distal openings 114A, 114B, and is slidably engaged with one or both of the first guidewire 210A and the second guidewire 210B. For example, a portion of the first guidewire 210A is slidably engaged with the first aperture 222A and a portion of the second guidewire 210B is slidably engaged with the second aperture 222B of the link 220.

A lateral distance between the first aperture 222A and the second aperture 222B is such that, as the link 220 is positioned adjacent the distal openings 114A, 114B, the portion of the first guidewire 210A is urged towards the portion of the second guidewire 210B, which in turn elastically deforms the first split tip end 112A to be positioned adjacent the second split tip end 112B in a placement configuration. As noted, a cross-sectional shape of the first split tip end 112A and the second split tip end 112B can each define a semi-circular shape. As such, when elastically deformed to align with each other, the first split tip end 112A and the second split tip end 112B can substantially define a circular outer profile, similar to a circular outer profile of a proximal portion of the catheter body 102.

In an embodiment, the first guidewire 210A includes a stop feature 230 such as a barb, pawl, or the like. In an embodiment, the barb 230 is formed integrally with the first guidewire 210A as a single monolithic piece. In an embodiment, the barb 230 is formed as a separate structure, and optionally formed of a different material, from that of the first guidewire 210A. The barb 230 can then be coupled with the first guidewire 210A using adhesive, bonding, welding, ultrasonic welding, or the like.

In an embodiment, the barb 230 is configured to slidably engage the first aperture 222A of the link 220 in a first direction and configured to prevent movement of the link 220 relative to the first guidewire 210A in a second direction. For example, as shown, the barb 230 is a frusto-conical shape extending radially from the first guidewire 210A and includes a tapered surface 232 facing distally, and an abutment surface 234 facing proximally. An outer diameter of the barb 230 can be equal to or slightly larger than an inner diameter of the first aperture 222A. In an embodiment, the barb 230 can be urged distally through the first aperture 222A in a first direction. However, when the barb 230 is urged proximally through the first aperture 222A, the abutment surface 234 engages a portion of the link 220 and prevents further proximal movement of the first guidewire 210A relative to the link 220. As such, urging the first guidewire 210A proximally can also urge the link 220 proximally towards distal end of the catheter 100 and elastically deforming the split tip feature 110.

In an exemplary method of use, a catheter 100 is provided as disclosed herein. A first guidewire 210A including a barb 230 disposed at a distal end is advanced proximally through a first lumen 104A. A second guidewire 210B is advanced proximally through a second lumen 104B. A link 220 is positioned distally of the distal end of the catheter 100 and the barb 230 is urged through a first aperture 222A of the link 220. A distal end of the second guidewire 210B is slidably engaged with the second aperture 222B of the link 220. The first guidewire 210A is then withdrawn proximally. The abutment surface 234 of the barb 230 engages a portion of the link 220 and urges the link 220 towards the first distal opening 114A of the first lumen 104A. The second aperture 222B slidably engages the second guidewire 210B and is urged towards the second distal opening 114B of the second lumen 104B. Due to the distance between the first aperture 222A and the second aperture 222B of the link 220, the first distal opening 114A is urged towards the second distal opening 114B elastically deforming the Y-shaped split tip feature 110 to a placement configuration, as described herein. The split tip feature 110 can then be advanced over the second guidewire 210B towards a target location within the vasculature.

Once the split tip feature 110 is placed at a target location within the vasculature, the second guidewire 210B can be withdrawn proximally through the second lumen 104B to disengage the second aperture 222B of the link 220. The first guidewire 210A, barb 230 and link 220 assembly can then be urged proximally through the first lumen 104A to disengage the catheter 100. In an embodiment, a diameter of the link 220 is equal to, or less than an inner diameter of the first lumen 104A allowing the link 220 to be urged through the first lumen 104A with little or no elastic distortion of the first lumen 104A. In an embodiment, the link 220 is formed of a flexible material that allows the link 220 to elastically deform to first through the first lumen 104A. The split tip feature 110 then resumes the elastically undeformed Y-shape. Advantageously, the link catheter placement system 200 provides a system of placing a split tip catheter that is easy to manufacture and assemble providing associated cost savings and manufacturing efficiencies.

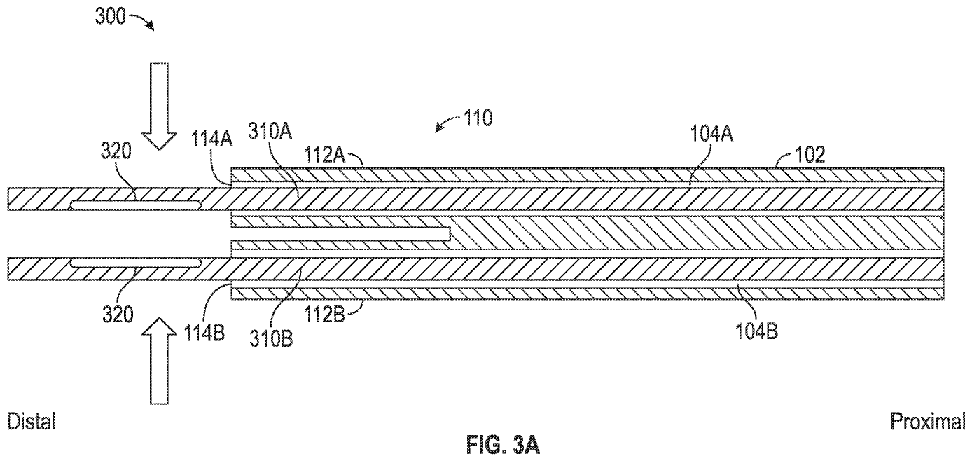

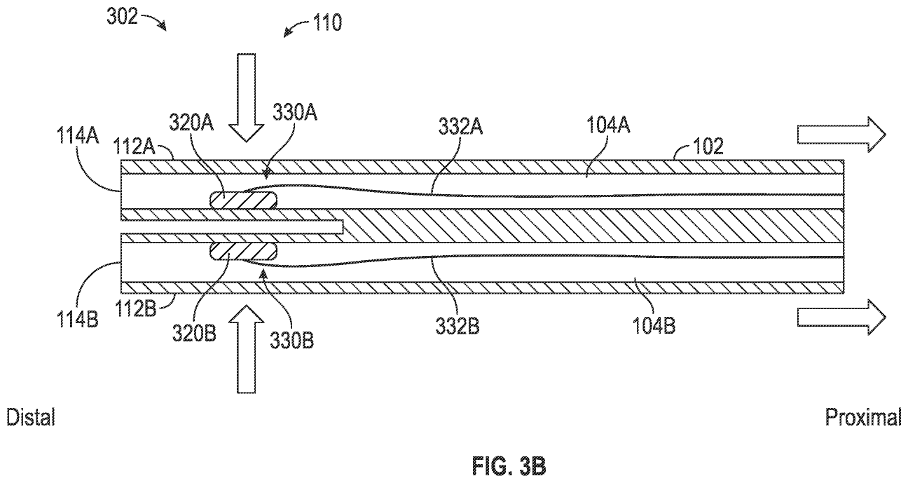

FIG. 3A shows an embodiment of a magnetic catheter placement system 300 generally including a catheter 100 as described herein, a first guidewire 310A and a second guidewire 310B. One or both of the first guidewire 310A and the second guidewire 310B includes a magnetic element 320. As used herein a magnetic element 320 includes a permanent magnet, a magnetic material, or a magnetizable material. The magnetic element 320 can be disposed at a distal end of the guidewire 310. As such, when the distal end of the first guidewire 310A and the distal end of the second guidewire 310B are advanced distally of the distal openings 114A, 114B, the magnetic element(s) 320 can attract the distal end of the first guidewire 310A to engage the distal end of the second guidewire 310B. The first guidewire 310A and the second guidewire 310B can then be withdrawn proximally through the respective first lumen 104A and second lumen 104B to elastically deform the first split tip end 112A and the second split tip end 112B to a placement configuration. Further, the distal end of the first guidewire 310A engaged with the distal end of the second guidewire 310B and the split tip feature 110 in a placement configuration forms a tapered outer profile to the assembly facilitating negotiating tortuous vascular pathways.

The split tip feature 110 in the placement configuration can then be advanced through the vasculature to a target location. Once at the target location, one or both of the first guidewire 310A and the second guidewire 310B can then be urged proximally until the distal end of the first guidewire 310A disengages from the distal end of the second guidewire 310B allowing the split tip feature 110 to resume the Y-shape configuration. The first guidewire 310A can then be withdrawn proximally through the first lumen 104A and the second guidewire 310B can then be withdrawn proximally through the second lumen 104B to disengage the catheter 100. Advantageously, the magnetic catheter placement system 300 provides a system of placing a split tip catheter that is easy to manufacture and assemble providing associated cost savings and manufacturing efficiencies.

FIG. 3B shows another embodiment of a magnetic catheter placement system 302 generally including a catheter 100 as described herein, a first tethered magnet 330A and a second tethered magnet 330B. The first tethered magnet 330A and a second tethered magnet 330B each include a magnetic element 320 (i.e., a first magnetic element 320A and a second magnetic element 320B respectively), as described herein, and a tether 332 (i.e., a first tether 332A and a second tether 332B respectively) coupled to the magnetic element 320 and extending proximally. The tether can be formed of a plastic, polymer, elastomer, rubber, silicone rubber, silk, nylon, polytetrafluoroethylene (PTFE), expanded polytetrafluoroethylene (ePTFE), polyurethane (PU), polyether block amide (PEBA; PEBAX®), or the like. In an embodiment the tether 330 can be formed of one or more strands of woven, braided, or coiled material including a plastic, polymer, nylon, metal, alloy, shape-memory material, super-elastic material, nitinol, composite, or the like and can be formed as a single strand, woven or twisted strands, flexible wire, or the like.

The first magnetic element 320A of the first tethered magnet 330A is placed within the lumen of the first split tip end 112A, adjacent the first distal opening 114A, and the second magnetic element 320B of the second tethered magnet 330B is placed within the lumen of the second split tip end 112B, adjacent the second distal opening 114B. The first tether 332A and the second tether 332B can extend proximally to a proximal end of the catheter 100.

The first magnetic element 320A and the second magnetic element 320B are attracted to each other through the walls of the first split tip end 112A and the second split tip end 112B and elastically deform the split tip feature from the Y-shape to the placement configuration. The catheter 100 can then be advanced to a target location within the vasculature. Optionally, the catheter 100 and magnetic catheter placement system 302 can be advanced over a guidewire to a target location. Once at the target location, one or both of the first tether 332A and the second tether 332B can be urged proximally to disengage the first magnetic element 320A from the second magnetic element 320B. The split tip feature can then transition from the placement configuration to the Y-shape. The first tethered magnet 330A and the second tethered magnet 330B can be removed proximally from the catheter 100 through the first lumen 104A and the second lumen 104B respectively. Advantageously, the magnetic catheter placement system 302 provides a system of placing a split tip catheter that is easy to manufacture and assemble providing associated cost savings and manufacturing efficiencies.

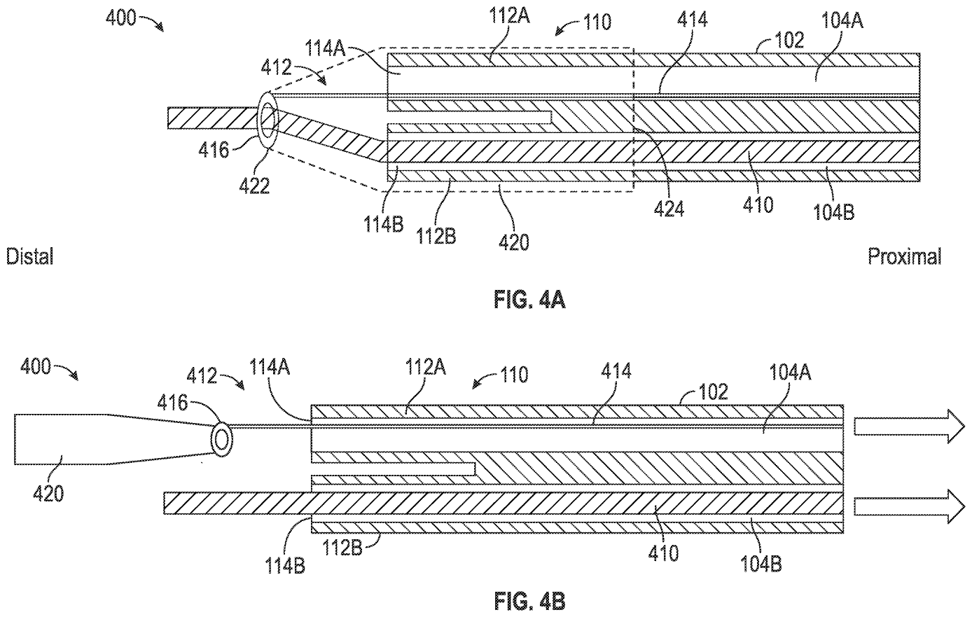

FIGS. 4A-4B show an embodiment of a sheath catheter placement system 400 that generally includes a catheter 100 having a split tip feature 110, a guidewire 410, a rod 412 and a sheath 420. The rod 412 includes a shaft 414 extending longitudinally, and a loop 416 coupled to a distal end of the shaft 414 and extending at an angle relative to the longitudinal axis of the shaft. In an embodiment, the plane of the loop 416 extends perpendicular to the axis of the shaft 414, however, other angles relative to the shaft 414 are also contemplated. The rod 412 is disposed within the first lumen 104A and extends distally of the distal end of the catheter 100 such that the loop 416 is disposed distally of the first distal opening 114A. The loop 416 defines an aperture configured to receive a portion of the guidewire 410 extending therethrough.

The guidewire 410 is disposed in the second lumen 104B with a distal portion of the guidewire 410 extending distally of the second distal opening 114B. In an embodiment, a distal portion of the guidewire 410 extends through the aperture of the loop 416. The guidewire 410 engaged with the rod 412 co-operate to elastically deform the split tip feature 110 to a placement configuration, i.e. the first split tip end 112A is aligned with the second split tip end 112B to define a substantially circular outer profile.

In an embodiment, the rod 412 further includes a sheath 420 coupled thereto. In an embodiment, the sheath 420 is formed of a flexible, or elastically deformable material, including a plastic, polymer, elastomer, rubber, silicone rubber, silk, nylon, polytetrafluoroethylene (PTFE), expanded polytetrafluoroethylene (ePTFE), polyurethane (PU), polyether block amide (PEBA; PEBAX®), or the like. In an embodiment the sheath is formed of a plurality of woven, braided, or coiled strands including a plastic, polymer, nylon, metal, alloy, shape-memory material, super-elastic material, nitinol, composite, or the like. In an embodiment, the sheath is formed of a wire coil extending in a conical and/or cylindrical shape. A first end 422 of the sheath 420 is coupled to the loop 416 and extends annularly about an outer edge thereof. The sheath 420 extends conically and/or cylindrically from the first end 422, along a longitudinal axis, to an opening disposed at a second end 424 opposite the first end 422. The sheath 420 can be formed of a flexible material such as a woven or non-woven plastic, polymer, elastomer, or the like. In an embodiment, the sheath 420 can be invertible between a first position (FIG. 4A) and a second position (FIG. 4B). The sheath 420 can be bistable in either the first position or the second position.

In an embodiment, an inner diameter of the sheath 420 at the second end 424 is equal to, or slightly larger than, the outer diameter of the catheter body 102. In an embodiment, the inner diameter of the sheath 420 can be less than outer diameter of the catheter body 102. In an embodiment, the inner diameter of the sheath 420 in the first position, in an undeformed state is equal to, or slightly larger than, the outer diameter of the loop 416 and can be elastically deformed radially outwards to the outer diameter of the catheter body 102.

As shown in FIG. 4A, in the first position, the sheath 420 extends proximally from the loop 416 such that the second end 424 is disposed proximally of the first end 422. A portion of the sheath 420 encircles the first split tip end 112A and the second split tip end 112B to retain the first split tip end 112A and the second split tip end 112B in a placement configuration. The guidewire 410 extends through second distal opening 114B and through the aperture defined by the loop 416 such that the distal tip of the guidewire 410 extends distally of the loop 416 to a target location within the vasculature of the patient. The catheter 100, rod 412 and sheath 420 assembly, with the sheath 420 in the first position, can be advanced over the guidewire 410 to the target location within the vasculature.

As shown in FIG. 4B, with the split tip feature 110 placed at the target location, the guidewire 410 can be withdrawn proximally so that the distal tip of the guidewire 410 passes through the aperture of the loop 416, through the second distal opening 114B and is removed proximally through the second lumen 104B. The rod 412 and sheath 420 can then be urged distally to disengage the sheath 420 from the split tip feature 110, before being urged proximally through the first lumen 104A. As the loop 416 passes through the first distal opening 114A, the sheath 420 can be inverted and transitioned to a second position (FIG. 4B) where the second end 424 of the sheath 420 is disposed distally of the first end 422. In an embodiment, the outer diameter of the loop 416 is equal to or less than the inner diameter of the first lumen 104A. In an embodiment, the loop 416 can define a circular or elliptical shape. In an embodiment, the joint between the loop 416 and the shaft 414 can be flexible, and/or hingedly engaged, to allow the plane of the loop 416 to move or elastically deform relative to the longitudinal axis of the shaft 414 and facilitate passing the loop 416 through the first lumen 104A.

As the sheath 420 disengages the split tip feature 110, the first split tip end 112A and the second split tip end 112B are released and allowed to resume the undeformed, Y-shape of the split tip feature 110 at rest. The rod 412 and sheath 420 assembly can then be withdrawn proximally through the first lumen 104A. To note, the sheath 420 is sufficiently flexible to collapse and fold, in order to fit within the first lumen 104A with little to no elastic distortion of the first lumen 104A. For example, where the sheath 420 is formed of a wire coil, the coil can unravel to allow the sheath 420 to be withdrawn through the first lumen 104A.

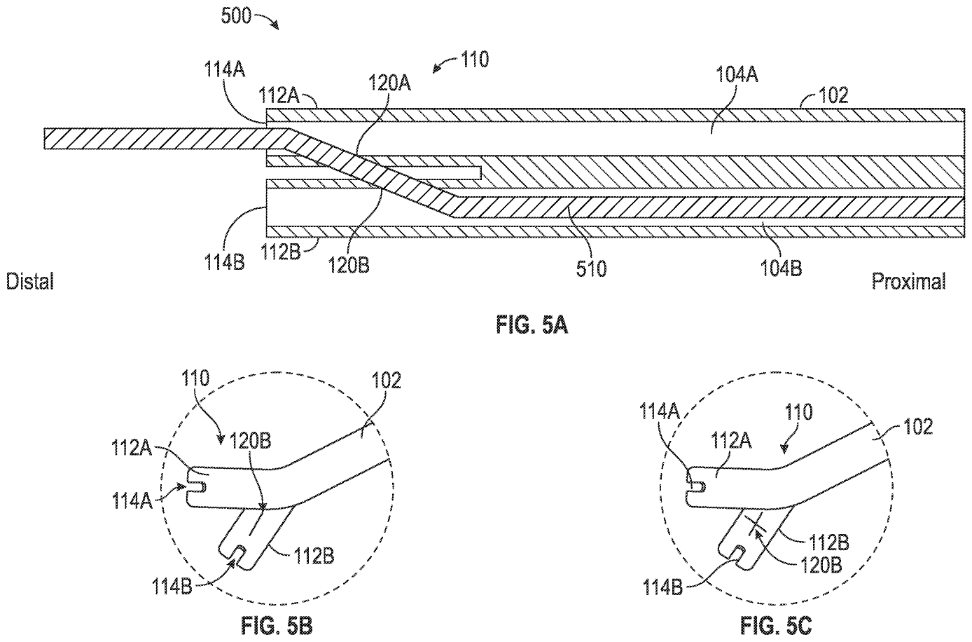

FIGS. 5A-5C show an embodiment of a threaded catheter placement system 500 generally including a catheter 100 and a guidewire 510. In an embodiment, the first split tip end 112A includes a first self-closing slit 120A disposed in a side wall of the first split tip end 112A and the second split tip end 112B includes a second self-closing slit 120B disposed in a side wall of the second split tip end 112B. Both the first self-closing slit 120A and the second self-closing slit 120B are configured to elastically deform to an open position to receive a portion of the guidewire 510 therethrough. In an embodiment, with the guidewire 510 removed from the first slit 120A and the second slit 120B, the first slit 120A and the second slit 120B resume an elastically undeformed, or closed, position. In the closed position, a first edge of the slit 120 (i.e., the first slit 120A and/or the second slit 120B) contacts a second edge, disposed opposite the first edge, to form a fluid tight seal therebetween when the slit 120 is in the closed position (FIG. 5B). In an embodiment, one or both of the first self-closing slit 120A and the second self-closing slit 120B can include one or more chamfers, bevels, skives, grooves or the like to facilitate guiding the guidewire 510 through the first self-closing slit 120A and/or the second self-closing slit 120B.

In an embodiment, as shown in FIG. 5B, the slit 120 (i.e., the first slit 120A and/or the second slit 120B) can define a linear shape, however, it will be appreciated that the slit 120 can include other shapes including one or more slit lines intersecting. For example, a cross shape (FIG. 5B) where two slit lines intersect, or a star shape where three or more slit lines intersect, each of which form a fluid tight seal when in the closed position.

With continuing reference to FIG. 5A, in an embodiment, the guidewire 510 engages the catheter 100 to elastically deform the split tip feature 110 to define a placement configuration, as described herein. A proximal portion of the guidewire 510 extends through the second lumen 104B. A distal portion of the guidewire 510 extends from the second lumen 104B, through the second slit 120B, through the first slit 120A and extends through a distal portion of the first lumen 104A. In an embodiment, a distal tip of the guidewire 510 extends through the first distal opening 114A and distally of the distal end of the catheter 100.

In an embodiment, the opposite configuration is also contemplated where a proximal portion of the guidewire 510 extends through the first lumen 104A. A distal portion of the guidewire 510 extends from the first lumen 104A, through the first slit 120A, through the second slit 120B and extends through a distal portion of the second lumen 104B. In an embodiment, a distal tip of the guidewire 510 extends through the second distal opening 114B and distally of the distal end of the catheter 100.

With the guidewire 510 threaded through both the first slit 120A and the second slit 120B, the guidewire 510 retains the split tip feature in the elastically deformed, placement configuration, as described herein. The catheter 100 and guidewire 510 assembly can then be advanced to a target location within the vasculature. Once placed, the guidewire 510 can be withdrawn proximally from the catheter 100 disengaging both the first slit 120A and the second slit 120B. The split tip feature 110 then resumes the elastically undeformed shape, as described herein. Further, the first slit 120A and/or the second slit 120B can resume the closed position where opposite edges of the slit contact each other to define a fluid tight seal therebetween. Advantageously, the slit(s) 120A, 120B, in the closed position prevent fluid from exiting the lumen prematurely, and maintain the fluid flow characteristics established by the first distal opening 114A and the second distal opening 114B as the fluid exits/enters the first lumen 104A and/or the second lumen 104B. Further, the slit(s) 120A, 120B, in the closed position mitigate the formation of biofilms, fibrin sheaths, thrombosis, or similar occlusions that can affect the useful life of the catheter 100.

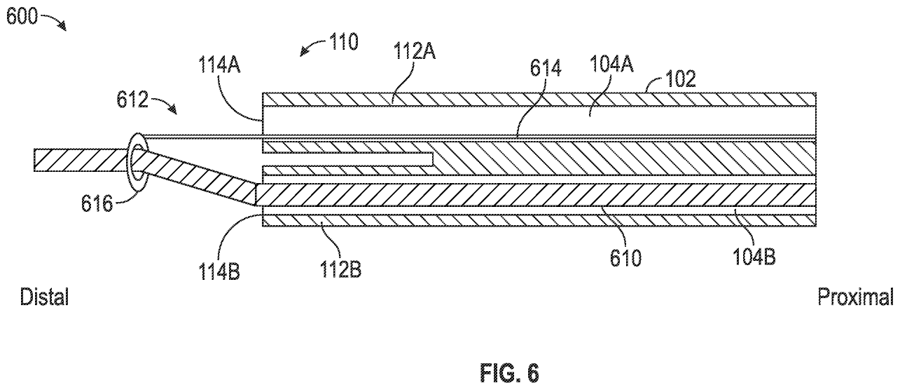

FIG. 6 shows an embodiment of a snare catheter placement system 600 that generally includes a catheter 100 having a split tip feature 110, a guidewire 610, and a snare 612. The snare 612 includes a shaft 614 extending longitudinally, and a loop 616 coupled to a distal end of the shaft 614 and extending at an angle relative to the longitudinal axis of the shaft 614. In an embodiment, the snare 612 is disposed within a first lumen 104A and extends distally of the catheter 100 such that the loop 616 is disposed distally of the first distal opening 114A. The loop 616 defines an aperture configured to receive a portion of the guidewire 610 extending therethrough.

In an embodiment, the guidewire 610 is disposed in the second lumen 104B with a distal portion thereof extending distally of the second distal opening 114B. A distal portion of the guidewire 610 extends through the aperture of the loop 616 and elastically deforms the split tip feature 110 to the placement configuration, such that the first split tip end 112A is aligned with the second split tip end 112B to define a substantially circular outer profile. The catheter 100 and snare 612 assembly can then be advanced over the guidewire 610 to the target location within the vasculature.

Once at the target location, the guidewire 610 can be withdrawn proximally so that the distal tip of the guidewire 610 passes through the aperture of the loop 616, to disengage the snare 612 allowing the split tip feature 110 to resume the Y-shape. The guidewire 610 is then removed proximally through the second lumen 104B. The snare 612 is then also withdrawn proximally through the first lumen 104A.

In an embodiment, the outer diameter of the loop 616 is equal to or less than the inner diameter of the first lumen 104A. In an embodiment, the loop 616 can define a circular or elliptical shape. In an embodiment, the joint between the loop 616 and the shaft 614 can be flexible, and/or hingedly engaged, to allow the plane of the loop 616 to move or elastically deform relative to the longitudinal axis of the shaft 614 and allow the snare 612 to pass through the first lumen 104A. As the guidewire 610 disengages the snare 612, the first split tip end 112A and the second split tip end 112B are released and allowed to resume to the undeformed, Y-shape of the split tip feature 110 at rest.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A catheter placement system, comprising:
a split tip catheter having a catheter body extending along a longitudinal axis and defining a first lumen and a second lumen, and a split tip feature comprising:
a first split tip end extending at a first angle relative to the longitudinal axis and including a distal portion of the first lumen encircled by a first outer wall; and
a second split tip end extending at a second angle relative to the longitudinal axis and including a distal portion of the second lumen encircled by a second outer wall;
a first guidewire disposed in the first lumen and including a stop feature disposed on a distal portion thereof;
a second guidewire disposed in the second lumen; and
a link defining a first aperture configured to receive a portion of the first guidewire, and a second aperture configured to receive a portion of the second guidewire.

2. The catheter placement system according to claim 1, wherein the stop feature includes an abutment surface configured to engage the link and retain the link to the first guidewire.

3. The catheter placement system according to claim 2, wherein the stop feature includes a barb having a tapered surface facing distally and the abutment surface facing proximally and configured to slidably engage the first aperture of the link in a first direction and to prevent the first guidewire disengaging the first aperture of the link in a second direction.

4. The catheter placement system according to claim 1, wherein the link, engaged with both the first guidewire and the second guidewire, elastically deforms the split tip feature to a placement configuration where the first split tip end and the second split tip end extend substantially parallel to the longitudinal axis.

5. The catheter placement system according to claim 1, wherein the first angle extends in a first direction relative to the longitudinal axis, and the second angle extends in a second direction relative to the longitudinal axis.

6. A method of placing a split tip catheter having a catheter body extending along a longitudinal axis and defining a first lumen and a second lumen, and a split tip feature including a first split tip end extending at a first angle and a second split tip end extending at a second angle, relative to the longitudinal axis, the method comprising:
slidably engaging a distal portion of a first guidewire with a first aperture of a link;
slidably engaging a distal portion of a second guidewire with a second aperture of the link, the first guidewire disposed within the first lumen and the second guidewire disposed within the second lumen of the split tip catheter;
withdrawing the first guidewire proximally through the first lumen;
engaging a stop feature disposed at a distal end of the first guidewire with the link to urge the link proximally towards the first split tip end;
elastically deforming the first split tip end and the second split tip end to extend substantially parallel with the longitudinal axis; and
advancing the split tip catheter over the second guidewire to a target location.

7. The method according to claim 6, further comprising:
withdrawing the second guidewire proximally to disengage the second aperture;
transitioning the first split tip end to extend at the first angle and the second split tip end to extend at the second angle;
withdrawing the first guidewire proximally through the first lumen; and
urging the link proximally through the first lumen.

8. The method according to claim 6, wherein the stop feature includes a tapered surface facing distally and is configured to slidably engage the first aperture of the link in a first direction, and an abutment surface facing proximally and configured to prevent the link from disengaging the first guidewire in a second direction, opposite the first direction.

9. The method according to claim 6, wherein the first angle extends between 45° and 60° in a first direction from the longitudinal axis, and the second angle extends between 45° and 60° in a second direction from the longitudinal axis, the second direction being opposite the first direction.

10. A catheter placement system, comprising:
a split tip catheter having a catheter body extending along a longitudinal axis and defining a first lumen and a second lumen, and a split tip feature comprising:
a first split tip end extending at a first angle relative to the longitudinal axis in a first direction and including a distal portion of the first lumen encircled by a first outer wall; and
a second split tip end extending at a second angle relative to the longitudinal axis in a second direction, opposite the first direction, and including a distal portion of the second lumen encircled by a second outer wall;
a first guidewire disposed in the first lumen, and including a first magnetic element disposed at a distal end thereof; and
a second guidewire disposed in the second lumen, wherein the first magnetic element secures a distal portion of the first guidewire to a distal portion of the second guidewire to elastically deform the first split tip end adjacent the second split tip end.

11. The catheter placement system according to claim 10, wherein the second guidewire includes a second magnetic element disposed at a distal end thereof.

12. The catheter placement system according to claim 11, wherein one or both of the first magnetic element or the second magnetic element includes a permanent magnet.

13. A method of placing a split tip catheter having a split tip feature including a first split tip end extending a first angle to a longitudinal axis of the split tip catheter, and a second split tip end extending at a second angle to the longitudinal axis, the method comprising:
placing a first guidewire in a first lumen of the split tip catheter, the first guidewire including a first magnetic element disposed at a distal end thereof, a distal tip of the first guidewire disposed distally of a distal tip of the first split tip end;
placing a second guidewire in a second lumen of the split tip catheter, a distal tip of the second guidewire disposed distally of a distal tip of the second split tip end;
engaging the first magnetic element with a distal portion of the second guidewire;
elastically deforming the first split tip end and the second split tip end to extend parallel to the longitudinal axis; and
advancing the first guidewire, the second guidewire and the split tip catheter to a target location.

14. The method according to claim 13, further comprising:
withdrawing the first guidewire proximally from the first lumen to disengage the first magnetic element from the distal portion of the second guidewire;
transitioning the first split tip end to extend at the first angle and the second split tip end to extend at the second angle; and
withdrawing the second guidewire proximally from the second lumen.

15. The method according to claim 13, wherein the first angle extends in a first direction from the longitudinal axis, and the second angle extends in a second direction from the longitudinal axis, opposite the first direction.

16. A catheter placement system, comprising:
a split tip catheter having a catheter body extending along a longitudinal axis and defining a first lumen and a second lumen, and a split tip feature comprising:
a first split tip end extending at a first angle relative to the longitudinal axis in a first direction and including a distal portion of the first lumen encircled by a first outer wall; and
a second split tip end extending at a second angle relative to the longitudinal axis in a second direction, opposite the first direction, and including a distal portion of the second lumen encircled by a second outer wall;
a first magnetic element disposed in the distal portion of the first lumen; and
a second magnetic element disposed in the distal portion of the second lumen and configured to magnetically attract the first magnetic element to elastically deform the first split tip end adjacent the second split tip end.

17. The catheter placement system according to claim 16, wherein the first magnetic element includes a first tether extending proximally through the first lumen, and the second magnetic element includes a second tether extending proximally through the second lumen.

18. A method of placing a split tip catheter having a catheter body defining a first lumen and a second lumen, and a split tip feature including a first split tip end extending a first angle to a longitudinal axis of the split tip catheter, and a second split tip end extending at a second angle to the longitudinal axis, the method comprising:
placing a first magnetic element with a lumen of the first split tip end;
placing a second magnetic element with a lumen of the second split tip end;
engaging the first magnetic element with the second magnetic element;
elastically deforming the first split tip end and the second split tip end to extend parallel to the longitudinal axis; and
advancing the first magnetic element, the second magnetic element and the split tip catheter to a target location.

19. The method according to claim 18, further comprising:
urging a first tether coupled to the first magnetic element proximally to withdraw the first magnetic element proximally from the first lumen;
transitioning the first split tip end to extend at the first angle and the second split tip end to extend at the second angle; and
urging a second tether coupled to the second magnetic element proximally to withdraw the second magnetic element proximally from the second lumen.

20. The method according to claim 18, wherein the first angle extends in a first direction from the longitudinal axis, and the second angle extends in a second direction from the longitudinal axis, opposite the first direction.

21-44. (canceled)
